(19) United States
(12) Patent Application Publication
(10) Pub. No.: US 2023/0301632 A1
(43) Pub. Date: Sep. 28, 2023

(54) ULTRASONIC IMAGING METHOD, ULTRASONIC IMAGING APPARATUS AND STORAGE MEDIUM

(71) Applicant: SHENZHEN MINDRAY BIO-MEDICAL ELECTRONICS CO., LTD., Shenzhen (CN)

(72) Inventors: Chongchong GUO, Shenzhen (CN); Lei LI, Shenzhen (CN); Jing LIU, Shenzhen (CN); Bo YANG, Shenzhen (CN)

(21) Appl. No.: 18/190,756

(22) Filed: Mar. 27, 2023

(30) Foreign Application Priority Data
Mar. 25, 2022 (CN) 202210306956.9

(51) Int. Cl. A61B 8/08; A61B 8/00

(52) U.S. Cl. CPC A61B 8/52; A61B 8/44

(57) ABSTRACT

An ultrasonic imaging method includes: controlling an ultrasonic probe to transmit ultrasonic waves to a biological tissue under examination and receive echo signals of the biological tissue under examination, the echo signals comprising one group of channel data; beam-forming the channel data by using a first beam-forming procedure to obtain beam-formed data of a first group of beam-forming points, and generating a first ultrasonic image of the biological tissue under examination based on the beam-formed data of the first group of beam-forming points; determining a region of interest based on the first ultrasonic image; beam-forming the channel data by using a second beam-forming procedure to obtain beam-formed data of a second group of beam-forming points; generating a second ultrasonic image of the region of interest based on the beam-formed data of the second group of beam-forming points; and displaying the first and second ultrasonic images in a fusion manner.

### CROSS-REFERENCE TO RELATED APPLICATIONS

This application is based on and claims priority to and benefits of Chinese Patent Application No. 202210306956.9, filed on Mar. 25, 2022. The entire content of the above-referenced application is incorporated herein by reference.

### TECHNICAL FIELD

The present disclosure relates to ultrasonic imaging, in particular to ultrasonic imaging methods, ultrasonic imaging apparatus and storage media.

### BACKGROUND

Medical ultrasonic imaging, in which ultrasonic echo signals are used to detect the structure of human tissue information, has the advantages of non-invasion, low cost and real-time display, resulting in a more and more wide application in clinical.

An ultrasonic system generally includes an ultrasonic probe, a transmitting/receiving control circuit, a processing circuit for beam-forming and signal processing, and a display. The beam-forming unit determines an overall image level, and different beam-forming procedures have different effects on images. A single beam-forming procedure is used for an entire imaging region to generate ultrasonic images in current mainstream medical ultrasonic imaging; that is, the same beam-forming procedure is adopted for imaging within the entire imaging region, and parameters for beam-forming are optimized in a compromise way, so as to maintain the uniformity of the images in the entire imaging region and make an overall image display effect in the imaging region be the best.

However, in the clinical application of ultrasound, the structural differences of many lesions are very small, especially in some difficult patients or difficult scenes. For example, small calcified lesions in blood vessels are difficult for doctors to make an accurate diagnosis of small lesions because they are relatively small, and the overall image optimization is often a goal in traditional ultrasound images without individual processing for small lesions. Some advanced beam-forming procedures in the industry can outline the boundary of lesions more clearly to enhance the contrast between tissue and normal tissue to help doctors to diagnose, but these methods often affect the overall image effect. How to balance the overall and local imaging effect in ultrasonic imaging is one of the problems to be solved or improved currently.

### SUMMARY

An ultrasonic imaging method provided in an embodiment may include:
controlling an ultrasonic probe to transmit ultrasonic waves to a biological tissue under examination and receive echoes from the biological tissue under examination to obtain multiple groups of channel data;
beam-forming the channel data at a first group of beam-forming points by using a first beam-forming procedure to obtain beam-formed data of the first group of beam-forming points, the first group of beam-forming points corresponding to respective location points in the biological tissue under examination in a space covered by the ultrasonic waves;
generating a first ultrasonic image of the biological tissue under examination according to the beam-formed data of the first group of beam-forming points;
determining a region of interest in the first ultrasonic image;
beam-forming the channel data at a second group of beam-forming points by using a second beam-forming procedure to obtain beam-formed data of the second group of beam-forming points, the second group of beam-forming points corresponding to respective location points in the region of interest;
generating a second ultrasonic image of the region of interest according to the beam-formed data of the second group of beam-forming points; and
displaying the first ultrasonic image and the second ultrasonic image in a fusion manner.

In an embodiment, said beam-forming the channel data at a second group of beam-forming points using a second beam-forming procedure to obtain beam-formed data of the second group of beam-forming points may include:
beam-forming the channel data at a third group of beam-forming points by using the second beam-forming procedure to obtain beam-formed data of the third group of beam-forming points, the third group of beam-forming points corresponding to respective location points in the biological tissue under examination in a space covered by the ultrasonic waves; and
selecting data corresponding to location points falling within the region of interest from the third group of beam-forming points as the beam-formed data of the second group of beam-forming points.

An ultrasonic imaging method provided in an embodiment may include:
controlling an ultrasonic probe to transmit first ultrasonic waves to a biological tissue under examination and receive echoes from the biological tissue under examination to obtain a first channel data;
beam-forming the first channel data at a first group of beam-forming points using a first beam-forming procedure to obtain beam-formed data of the first group of beam-forming points, the first group of beam-forming points corresponding to respective location points in the biological tissue under examination in a space covered by the ultrasonic waves;
generating a first ultrasonic image of the biological tissue under examination according to the beam-formed data of the first group of beam-forming points;
determining a region of interest based on the first ultrasonic image;
controlling the ultrasonic probe to transmit second ultrasonic waves to the region of interest and receive echoes from the region of interest to obtain a second channel data;
beam-forming the second channel data at a second group of beam-forming points using a second beam-forming procedure to obtain beam-formed data of the second group of beam-forming points, the second group of beam-forming points corresponding to respective location points in the region of interest;
generating a second ultrasonic image according to the beam-formed data of the second group of beam-forming points; and
displaying the first ultrasonic image and the second ultrasonic image in a fusion manner.

In an embodiment, before beam-forming the channel data at the first group of beam-forming points using the first beam-forming procedure, the method may further include:
obtaining an imaging setting of current ultrasonic imaging, and determining a beam-forming procedure matching the imaging setting from a plurality of predetermined beam-forming procedures as the first beam-forming procedure based on the imaging setting; or
displaying a plurality of beam-forming selection items on a display interface, each beam-forming selection item being associated with at least one beam-forming procedure; and detecting a first selection instruction generated based on a user's selection instruction on the beam-forming selection items to determine the first beam-forming procedure based on the first selection instruction.

In an embodiment, the imaging setting may include at least one of an ultrasonic probe type, a probe scan mode, a type of biological tissue under examination, and an imaging parameter for ultrasonic imaging.

In an embodiment, before said beam-forming the channel data at a second group of beam-forming points using a second beam-forming procedure, the method may further include:
determining the second beam-forming procedure from a plurality of predetermined beam-forming procedures based on a region image within the region of interest in the first ultrasonic image; or
displaying a plurality of beam-forming selection items on a display interface after determining the region of interest, each beam-forming selection item being associated with at least one beam-forming procedure; and detecting a second selection instruction generated based on a user's selection instruction on the beam-forming selection items to determine the second beam-forming procedure based on the second selection instruction.

In an embodiment, said determining the second beam-forming procedure from a plurality of predetermined beam-forming procedures based on a region image within the region of interest in the first ultrasonic image may include:
obtaining tissue information contained in the region image within the region of interest in the first ultrasonic image, and determining the second beam-forming procedure from the plurality of predetermined beam-forming procedures based on the tissue information.

In an embodiment, said determining the second beam-forming procedure from the plurality of predetermined beam-forming procedures based on the tissue information may include:
determining a beam-forming procedure being able to enhance boundary information as the second beam-forming procedure when the region image within the region of interest in the first ultrasonic image contains more tissue boundaries than small tissues; or
determining a beam-forming procedure being able to improve spatial resolution as the second beam-forming procedure when the region image within the region of interest in the first ultrasonic image contains more small tissues than tissue boundaries.

In an embodiment, the plurality of predetermined beam-forming procedures comprise at least two of a delay and sum (DAS) beam forming procedure, a minimum variance (MV) beam forming procedure, a coherent factor beam forming procedure, an incoherent beam forming procedure, and a frequency domain beam forming procedure.

In an embodiment, said fusing and displaying the first ultrasonic image and the second ultrasonic image may include:
overlaying the second ultrasonic image on the region of interest in the first ultrasonic image; or
segmenting a part outside the region of interest from the first ultrasonic image, and displaying the second ultrasonic image with the part outside the region of interest from the first ultrasonic image in a spliced manner.

In an embodiment, the first beam-forming procedure differs from the second beam-forming procedure in at least one of principles, steps and parameters.

In an embodiment, after fusing the first ultrasonic image and the second ultrasonic image, the method may further include:
determining a transition zone adjacent to the region of interest according to the region of interest, the transition zone surrounding the region of interest; and
filling the transition zone with pixel values in the region of interest to reduce a color difference between inside and outside boundaries of the region of interest.

An ultrasonic imaging apparatus provided in an embodiment may include:
an ultrasonic probe configured to transmit ultrasonic waves to a biological tissue under examination and receive echoes of the biological tissue under examination to obtain channel data;
a beam former configured to beam-forming the channel data to obtain beam-formed data;
a display configured to display an ultrasonic image; and
a processor configured to control the ultrasonic probe, the beam former and the display to perform any one of the ultrasonic imaging methods mentioned above.

In an embodiment, provided is a computer-readable storage medium having stored thereon a program that capable of being executed by a processor to implement any one of the methods mentioned above.

In the aforesaid embodiments, after obtaining the channel data, the beam-formed data of the first group of beam-forming points is generated by using the first beam-forming procedure, based on which the first ultrasonic image is generated to determine the region of interest, then the second beam-forming procedure different from the first beam-forming procedure is adopted to generate the second ultrasonic image corresponding to the region of interest, and finally the first and second ultrasonic images are fused and displayed. Accordingly, the whole imaging region and the region of interest (locally) can be performed with the most appropriate beam-forming procedure respectively by means of the above methods, which can take into account the imaging effect of the whole imaging region and local region of interest, providing users with better images.

### BRIEF DESCRIPTION OF THE DRAWINGS

FIG. 1 is a schematic diagram of an ultrasonic imaging apparatus according to an embodiment;

FIG. 2 is a schematic diagram of a display interface for selecting beam-forming procedures according to an embodiment;

FIG. 3 is a schematic diagram of fused images according to an embodiment;

FIG. 4 is a schematic diagram of fused images according to another embodiment;

FIG. 5 is a schematic diagram of fused images according to yet another embodiment;

FIG. 6 is a schematic diagram of the generation of a fusion image according to an embodiment;

FIG. 7 is a schematic diagram of fused images according to still yet another embodiment; and

FIG. 8 is a flowchart of an ultrasonic imaging method according to an embodiment.

### DETAILED DESCRIPTION

The present disclosure will be further described in detail below through specific embodiments with reference to the accompanying drawings. Common or similar elements are referenced with like or identical reference numerals in different embodiments. Many details described in the following embodiments are for better understanding the present disclosure. However, those skilled in the art can realize with minimal effort that some of these features can be omitted in different cases or be replaced by other elements, materials and methods. For clarity some operations related to the present disclosure are not shown or illustrated herein so as to prevent the core from being overwhelmed by excessive descriptions. For those skilled in the art, such operations are not necessary to be explained in detail, and they can fully understand the related operations according to the description in the specification and the general technical knowledge in the art.

In addition, the features, operations or characteristics described in the specification may be combined in any suitable manner to form various embodiments. At the same time, the steps or actions in the described method can also be sequentially changed or adjusted in a manner that can be apparent to those skilled in the art. Therefore, the various sequences in the specification and the drawings are only for the purpose of describing a particular embodiment, and are not intended to be an order of necessity, unless otherwise stated one of the sequences must be followed.

The serial numbers of components herein, such as “first”, “second”, etc., are only used to distinguish the described objects and do not have any order or technical meaning. The terms “connected”, “coupled” and the like here include direct and indirect connections (coupling) unless otherwise specified.

The most important idea of the invention is that two different beamforming methods are used for the entire imaging region and the region of interest, thereby allowing for both global and local imaging effects. In addition, the whole imaging region can be obtained by fusing one, two or more beamforming methods, and the region of interest can also be obtained by fusing one, two or more beamforming methods, but the whole imaging region is different from the region of interest in at least one beamforming method.

The most important idea of the invention is that two different beam-forming methods are used for the entire imaging region and the region of interest, thus taking into account the overall and local imaging effects. In addition, the entire imaging region can be fused by one, two or more beam-forming methods, and the region of interest can also be fused by one, two or more beam-forming methods, but the entire imaging region is different from the region of interest by at least one beam-forming method.

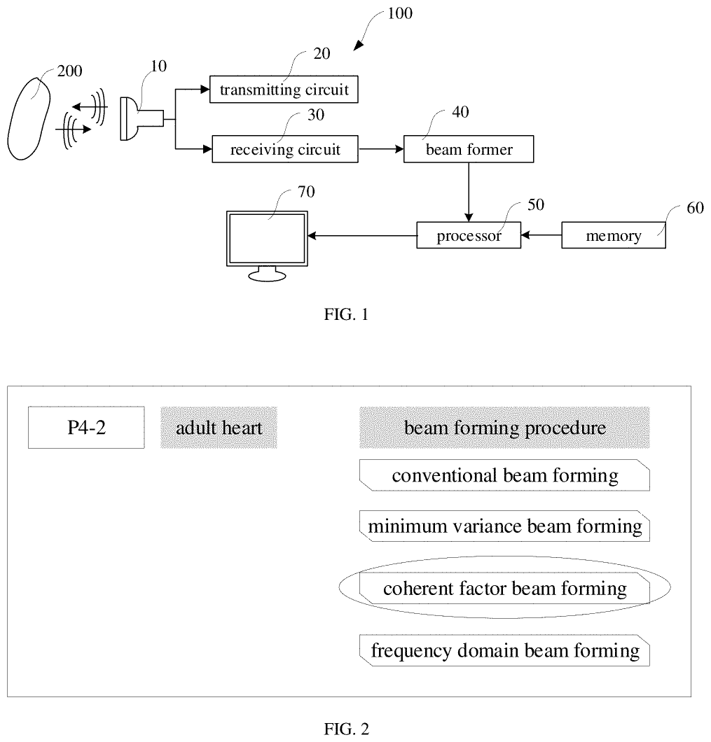

Referring to FIG. 1, there is provided an ultrasonic imaging apparatus 100 comprising an ultrasonic probe 10, a transmitting circuit 20, a receiving circuit 30, a beam former 40, a processor 50, a memory 60 and a human-computer interaction device 70.

The ultrasonic probe 10 may include a transducer (not shown in the figure) composed of a plurality of array elements, which are arranged into a row to form a linear array or a two-dimensional matrix to form a planar array. The plurality of array elements may also form a convex array. The array elements (for example using piezoelectric crystals) may convert electrical signals into ultrasonic signals in accordance with a transmission sequence transmitted by the transmitting circuit 20. The ultrasonic signals may, depending on applications, include one or more scanning pulses, one or more reference pulses, one or more impulse pulses and/or one or more Doppler pulses. According to the pattern of waves, the ultrasonic signals may include a focused wave, a plane wave and a divergent wave. The array elements may be configured to transmit an ultrasonic beam according to an excitation electrical signal or convert a received ultrasonic beam into an electrical signal. Each array element can accordingly be configured to achieve a mutual conversion between an electrical pulse signal and an ultrasonic beam, thereby achieving the transmission of ultrasonic waves to a biological tissue under examination 200, and can also be configured to receive ultrasonic echo signals reflected back by the tissue. During ultrasonic detection, the transmitting circuit 20 and the receiving circuit 30 can be used to control which array elements are used for transmitting the ultrasonic beam (referred to as transmitting array elements), which array elements are used for receiving the ultrasonic beam (referred to as receiving array elements), or to control the array elements to be used for transmitting the ultrasonic beam or receiving echoes of the ultrasonic beam in time slots. The array elements involved in transmission of ultrasonic waves can be excited by electric signals at the same time, so as to emit ultrasonic waves simultaneously; alternatively, the array element involved in transmission of ultrasonic waves can also be excited by a number of electric signals with a certain time interval, so as to continuously emit ultrasonic waves with a certain time interval. If the minimum processing area for receiving and reflecting ultrasonic waves in the biological tissue under examination 200 is referred to as a location point within the tissue, after reaching each location point of the biological tissue under examination 200, the ultrasonic waves may generate different reflections due to the different acoustic impedance of the tissue at different location points; then the reflected ultrasonic waves may be picked up by the receiving array elements, and each receiving array element may receive ultrasonic echoes of a plurality of location points. The ultrasonic echoes of different location points received by each receiving array element may form different channel data; and multiple channel data output by each receiving array element may form a set of channel data corresponding to the receiving array element. For a certain receiving array element, the distance from the receiving array element to different location points of the biological tissue under examination 200 is different, so the time when the ultrasonic echoes reflected by each location point reach the array element is also different; accordingly, a the corresponding relationship between the ultrasonic echoes and the location point can be identified according to the time when the ultrasonic echoes reach the array element.

In ultrasonic imaging, a frame of two-dimensional image is obtained by arranging several beam-forming points on the two-dimensional plane in sequence according to the spatial position relationship and conducting envelope detection, dynamic range compression and DSC, Digital Scan Conversion. The beam-forming point is the sum result of the data of each channel after phase compensation. The beam-forming point in this application corresponds to the position point mentioned above (for example, one-to-one or in other forms). The key of phase compensation is to determine the time sequence of ultrasonic echo arriving at each element, and the time sequence is determined by the spatial position (space distance divided by sound velocity equals time).

In ultrasonic imaging, a two-dimensional image of a frame is obtained after several beam combination points are sequentially arranged in a two-dimensional plane according to a spatial position relationship and operations such as envelope detection, dynamic range compression and digital scan conversion (DSC, digital Scan Conversion) are performed; the beam combination points are the results of summing data of each channel after phase compensation is performed; the beam combination points in the present application correspond to the above-mentioned position points (for example, one-to-one correspondence or corresponding in other forms), wherein the key of phase compensation is to determine the time sequence of ultrasonic echo arriving at each array element; the time sequence is determined by the spatial position (the spatial distance divided by the speed of sound equals time).

The transmitting circuit 20 may be configured to, depending on the control of the processor 50, generate the transmission sequence which may be configured to control some or all of the plurality of array elements to transmit ultrasonic waves to the biological tissue. Parameters of the transmission sequence may include the position(s) of transmitting array element(s), the number of the array elements, and the transmission parameter(s) of the ultrasonic beam (such as amplitude, frequency, number of transmissions, transmission interval, transmission angle, wave pattern, focus position, etc.). In some cases, the transmitting circuit 20 may also be configured to phase delay the transmitted beam so that different transmitting array elements transmit at different times, thereby each transmitting ultrasonic beam can be focused in a predetermined area. Due to different operating modes, such as B-image mode, C-image mode and D-image mode (Doppler mode), the parameters of the transmission sequence may be various.

The receiving circuit 30 may be configured to receive ultrasonic echo signals from the ultrasonic probe 10 and process the ultrasonic echo signals. The receiving circuit 30 may include one or more amplifiers, analog-to-digital converters (ADC), etc. The amplifier may be configured to amplify the received echo signals after appropriate gain compensation. The ADC may be configured to sample analog echo signals at a predetermined time interval to convert them into a digitized signal which still remains amplitude information, frequency information and phase information. The data output by the receiving circuit 30 may be output to the beam former 40 for processing or to the memory 60 for storage.

The beam former 40 in signal communication with the receiving circuit 30 may be configured to perform beam-forming on the echo signals. In this embodiment, A variety of beam-forming procedures may be pre-stored in the memory 60, including but not be limited to, a delayed and apodized summation (DAS) algorithm, a minimum variance (MV) beam forming procedure, a coherent factor beam forming procedure, a beam-forming procedure using filtered delayed multiplication and summation, etc.

The processor 50 may be configured as a central controller circuit (CPU), one or more microprocessors, graphics controller circuits (GPUs), or any other electronic component capable of processing input data in accordance with specific logic instructions. It may perform control of peripheral electronic components based on the input instructions or predetermined instructions, or perform data reading and/or storage from the memory 60, or perform processing of input data by executing programs in the memory 60. The processor 50 may control the operations of the transmitting circuit 20 and the receiving circuit 30, for example, controlling the transmitting circuits 20 and receiving circuits 30 work alternately or simultaneously. The processor 50 may also determine an appropriate operating mode according to a user's selection or a program setting to generate a transmission sequence corresponding to the current operating mode, and transmit the transmission sequence to the transmitting circuit 20, so that the transmitting circuit 20 adopts the appropriate transmission sequence to control the ultrasonic probe 10 to transmit the ultrasonic waves.

The processor 50 may also configured to be in signal communication with the beam former 40 to generate a corresponding ultrasonic image based on the signals or data output from the beam former 40. Specifically in the present embodiment, when the user adjusts the ultrasonic probe 10 to transmit ultrasonic waves to the biological tissue under examination 200, the ultrasonic waves may cover part or all of the biological tissue under examination 200 according to the position and/or angle of the ultrasonic probe 10 relative to the biological tissue under examination 200, the multiple groups of channel data can be obtained according to the echo signals received by the ultrasonic probe 10; the beam former 40 may first perform beam-forming on the channel data at the first group of beam-forming points by using the first beam-forming procedure to obtain the beam-formed data of the first group of beam-forming points, the first group of beam-forming points may correspond to respective location points in the biological tissue under examination 200 in a space covered by the ultrasonic waves (for example, in a one-to-one correspondence or in other manners); and the processor 50 may generate the first ultrasonic image of the biological tissue under examination 200 according to the beam-formed data of the first group of beam-forming points, that is, the first ultrasonic image is an ultrasonic image about a region generated by using the first beam-forming procedure, where the region is defined as an entire imaging region (FFOV) hereinafter.

As used herein, “channel data” may refer to data corresponding to a channel of the ultrasonic imaging apparatus (corresponding to one or more array elements) prior to beam-forming processing. For example, it can be either a radio frequency signal before demodulation, or a baseband signal after demodulation, and so on.

The first beam-forming procedure mentioned above is a beam-forming that is judged currently by the user or automatically judged by the ultrasonic imaging apparatus 100 to be most suitable for the imaging of the entire imaging region. In some embodiments, after the user sets the imaging setting of the ultrasonic imaging, the processor 50 may detect the imaging setting for current ultrasonic imaging, and according to the imaging setting of the current ultrasonic imaging, determine the first beam-forming procedure matching the imaging setting from a plurality of predetermined beam-forming procedures, that is, the system can automatically recommend a suitable beam-forming procedure. The imaging setting may include at least one of the ultrasonic probe type, the probe scan mode, the type of biological tissue under examination and the imaging parameter for ultrasonic imaging. The probe type may include but not be limited to high-frequency probe, low-frequency probe, one-dimensional probe, two-dimensional probe, etc. The probe scan mode may include but not be limited to a linear scan mode, a convex scan mode, a sector scan mode, a deflection scan mode, etc. The type of biological tissue under examination may include but not be limited to a small organ, nerve, heart, abdomen, muscle bone, etc. Imaging parameters may include but not be limited to frequency, aperture, focus, transmission line, receiving line, etc.

In addition to being completely automatically selected by the imaging apparatus, in some embodiments, the processor 50 may control the display 70 to display at least one beam-forming selection item on the display interface after detecting that the imaging setting of the ultrasonic imaging is completed, each beam-forming selection item is linked to a beam-forming procedure; and when detecting a first selection instruction generated based on the user's selection instruction on the beam-forming selection items, the processor 50 may invoke the selected first beam-forming procedure based on the first selection instruction. For example, as shown in FIG. 1, the user may select a current scene from the display interface as examining an adult heart, and the probe type is P4-2, the processor 50 may control the display 70 to display the relevant beam-forming items for the user to select.

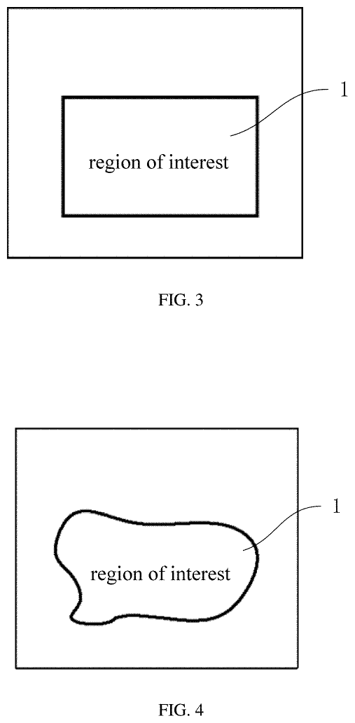

After obtaining the first ultrasonic image, a region of interest 1 can be determined in the first ultrasonic image according to the first ultrasonic image, and the region of interest 1 can be a part of an overlay of the first ultrasonic image. The shape of the region of interest 1 may be regular or irregular, e.g. regular in FIG. 3 and irregular in FIG. 4, and the way in which the region of interest 1 is determined may be automatic or non-automatic. The automatic way may include but not be limited to: the processor 50 determining the region of interest 1 in the first ultrasonic image by image recognition and other techniques; for example, performing feature extraction on the first ultrasonic image to obtain features of the entire image, and then performing matching detection on the features of the image to obtain one or more matched region as the region(s) of interest 1. The non-automatic way may include but not be limited to: selecting the region of interest 1 on the first ultrasonic image by a manual operation by the user; for example, selecting one or more regions of interest 1 by means of gestures, peripherals, voice controls, or the like from the first ultrasonic image which has been outputted on the display 70 by the processor 50.

After the region of interest 1 is determined, the processor 50 may control the beam former 40 to perform beam-forming on the channel data at the second group of beam-forming points by using the second beam-forming procedure to obtain the beam-formed data of the second group of beam-forming points corresponding to respective position points in the region of interest 1 on the biological tissue under examination 200 one by one. The first beam-forming procedure is different from the second beam-forming procedure. The difference therebetween may include that the first beam-forming procedure and the second beam-forming procedure are two completely different algorithms, for example, the first beam-forming procedure uses coherence factor while the second beam-forming procedure uses delayed multiplication and summation. The difference may further include that the first and second beam-forming procedures use the same technique but different synthesis parameters, for example, the first beam-forming procedure and the second beam-forming procedure both use a conventional DAS but adopt different window functions. For the conventional DAS method, an apodized window curve is usually preset, including a rectangular window, a Gaussian window, a Hanning window, a semi-circular window, etc. Different window functions can obtain different image effects, for example, the image corresponding to the rectangular window has a high spatial resolution but a lot of clutter, while the image corresponding to the Hanning window suppresses clutter but has a low spatial resolution; accordingly, the use of different window functions can also be considered as two different beam-forming procedures. In other words, the first beam-forming procedure and the second beam-forming procedure have at least one difference in principle, steps and parameters.

Similarly, as used herein, “different beam-forming procedures” or “a plurality of beam-forming procedures” may refer to the fact that the beam-forming procedures differ in at least one of the principles, steps and parameters, including different beam-forming procedures (principles), or the same algorithms (principles) with different steps therein (for example, increasing or decreasing steps or changing the sequence of steps, etc.), or different parameters used therein. The beam-forming procedures under such cases are considered to be “different” or “various” of beam-forming procedures.

The second beam-forming procedure mentioned above is suitable for the imaging of region of interest 1, which can well represent the details of region of interest 1. In some embodiments, the selection of the second beam-forming procedure may be similar to the first beam-forming procedure, i.e. the display 70 is controlled to display at least one beam-forming selection item for the user to select on the display interface, each beam-forming selection item is linked to a beam-forming procedure, a second selection instruction generated based on the user's selection instruction on the beam-forming selection items may be detected to invoke a second beam-forming procedure based on the second selection instruction. Different from the first beam-forming procedure, the beam-forming selection items displayed on the display interface can be determined based on the current imaging setting; alternatively, tissue structure within the region of interest 1 in the first ultrasonic image can be recognized after the determination of the region of interest 1, and the second beam-forming procedure can be determined from the plurality of predetermined beam-forming procedures based on the tissue structure within the region of interest 1.

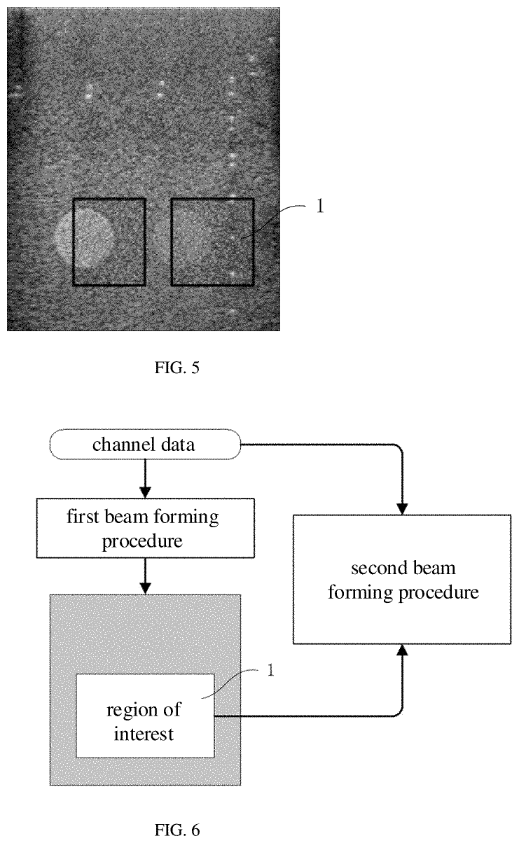

Furthermore, the ultrasonic imaging apparatus 100 may also determine the second beam-forming procedure from the plurality of predetermined beam-forming procedures based directly on the region image within the region of interest 1 in the first ultrasonic image, independent of the operation of the user. For example, the tissue information contained in the region image within the region of interest 1 in the first ultrasonic image may be obtained, and the second beam-forming procedure may be determined from the plurality of predetermined beam-forming procedures according to the tissue information contained in the region image within the region of interest 1 in the first ultrasonic image. For example, the region image within the region of interest 1 on the left in FIG. 5 contains predominantly tissue boundaries (e.g. contains more tissue boundaries than small tissues), then some beam-forming procedures that are able to enhance the boundary information of the tissue in the resultant ultrasound image can be used as the second beam-forming procedure. For another example, the region image within the region of interest 1 on the right in FIG. 5 contains predominantly small tissues (e.g. contains more small tissues than tissue boundaries), then the beam-forming procedures that are able to improve the spatial resolution of the resultant ultrasound image can be used as the second beam-forming procedure.

After obtaining the beam-formed data of the second group of beam-forming points, the processor 50 can generate the second ultrasonic image corresponding to the region of interest 1 according to the beam-formed data of the second group of beam-forming points, and then fuse the first ultrasonic image and the second ultrasonic image to obtain a fused image, that is, the ultrasonic image of the whole imaging region and the ultrasonic image of region of interest 1 are fused, thereby taking into account the overall and local imaging effects. When the display 70 displays the fused image, the user can not only grasp the entire imaging region from the original first ultrasonic image, but also better understand the characteristics of the tissue structure within the region of interest 1 based on the original second ultrasonic image.

In this example, the first ultrasonic image and the second ultrasonic image used for fusion are obtained based on the echo signals of the same ultrasonic waves, and the fusion process thereof is shown in FIG. 6. That is to say, after the ultrasonic probe 10 generates ultrasonic waves to the biological tissue under examination 200, channel data is obtained according to the echo signals. The channel data can be stored in the memory 60 in addition to the beam-formed data of the first group of beam-forming points. After determining the region of interest 1, the beam-formed data of the second group of beam-forming points is obtained according to the same channel data.

In some embodiments, the process of obtaining the beam-formed data of the second group of beam-forming points may be as follows: beam-forming the channel data at a third group of beam-forming points by using the second beam-forming procedure to obtain beam-formed data of the third group of beam-forming points, the third group of beam-forming points corresponding to respective location points in the biological tissue under examination 200 in a space covered by the ultrasonic waves; and selecting data corresponding to location points falling within the region of interest 1 from the third group of beam-forming points as the beam-formed data of the second group of beam-forming points. That is to say, the channel data is synthesized by the second beam-forming procedure first, and then the data of the beam-forming points corresponding to the location points in the region of interest 1 is selected as the beam-formed data of the second group of beam-forming points.

In other embodiments, after the region of interest 1 is determined, it is also possible to transmit new ultrasonic waves to the region of interest 1. To avoid confusion, the ultrasonic wave transmitted to the entire imaging region (the biological tissue under examination 200) is referred to as the first ultrasonic wave, the echo of the first ultrasonic wave is referred to as the first echo signal, the channel data extracted from the first echo signal is referred to as the first channel data, then the beam-formed data of the first group of beam-forming points is obtained by beam-forming of the first channel data at the first group of beam-forming points; whilst the ultrasonic wave re-emitted to the region of interest 1 is referred to as the second ultrasonic wave, the ultrasonic probe 10 may receive the second echo signal returned by the biological tissue under examination 200 in the region of interest 1 after the second ultrasonic wave is transmitted to the region of interest 1, the second channel data can be extracted from the second echo signal and then be beam synthesized at the second group of beam-forming points by using the second beam-forming procedure to obtain the beam-formed data of the second group of beam-forming points; and finally, the processor 50 generates a second ultrasonic image according to the beam-forming data of the second group of beam-forming points. It can thus be seen that, in other embodiments, the user may re-transmit the second ultrasonic wave for the region of interest 1, and the angle and direction of the second ultrasonic wave can be different from those of the first ultrasonic wave, so the second ultrasonic wave that is more appropriate for the region of interest 1 may be selected to further increase the imaging effect.

Furthermore, how to transmit the second ultrasonic wave can be determined according to the region of interest 1. In some embodiments, after determining the region of interest 1, the processor 50 may calculate data about the length and width of the region of interest 1, determine the transmitting array elements and the transmitting beam parameters according to the data about the length and width of the region of interest 1, and then control the transmitting array elements on the ultrasonic probe 10 to transmit the second ultrasonic wave to the region of interest 1 according to the transmitting beam parameters.

In some embodiments, the fusion between the first ultrasonic image and the second ultrasonic image is a “segmented” fusion which may be implemented in the following specific ways. One way may be to directly overlay the second ultrasonic image on the region of interest 1 in the first ultrasonic image, so that the region of interest 1 may directly display the second ultrasonic image. Another way may be to segment the part other than the region of interest 1 from the first ultrasonic image, and splice the second ultrasonic image with the part other than the region of interest 1 in the first ultrasonic image to obtain the fused image.

In other embodiments, the fusion between the first ultrasonic image and the second ultrasonic image is a “non-segmented” fusion. For example, the following formula may be used to fuse the first may image and the second may image to obtain the fused image:

P(i)ₒᵤₜₚᵤₜ=a(i)_{local}×P(i)_{local}+β(i)_{FFOV}×P(i)_{FFOV},i=1,2 . . . N

where P(i)_{local} represents a pixel value corresponding to the ith location point in the second ultrasonic image, α(i)_{local} a second fusion coefficient corresponding to the ith location point, P(i)_{FFOV} represents a pixel value corresponding to the ith location point in the first ultrasonic inane, β(i)_{FFOV} is a first fusion coefficient corresponding to the ith location point, and P(i)ₒᵤₜₚᵤₜ represents a pixel value corresponding to the ith location point in the fusion image. Generally, the sum of the fusion coefficient α(i)_{local} and β(i)_{FFOV} is 1, but it can also be other values. The fusion coefficient may be either a real number or a complex number; and one of the two fusion coefficients may be 0 or 1, or both may be 0 or 1. When the second fusion coefficient is 1 and the first fusion coefficient is 0, the imaging effect is consistent with the above-mentioned “segmented” fusion image.

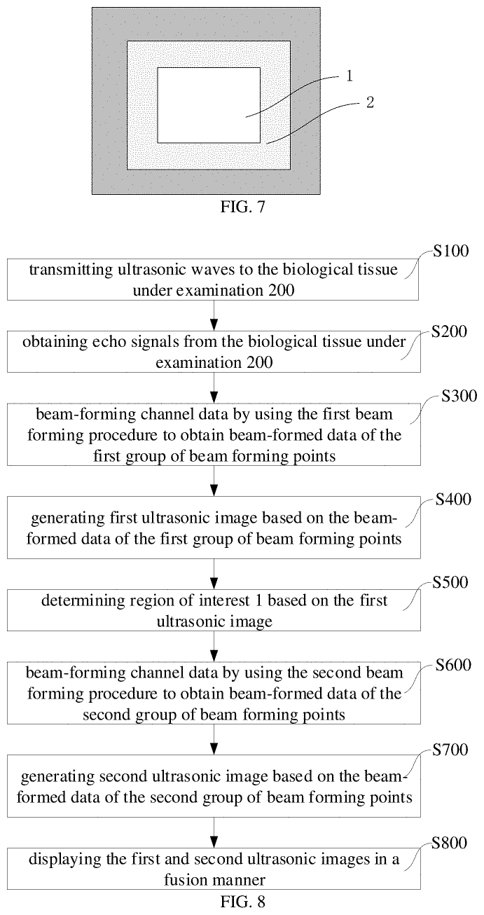

In some embodiments, as shown in FIG. 7, in order to make the boundary of the region of interest 1 more natural, a transition zone 2 may also be generated around the region of interest 1, and the pixel values within the transition zone 2 may be filled according to the pixel values in the region of interest 1. For example, an average or median value of the pixel values within the entire region of interest 1 may be obtained and then the transition zone 2 may be filled with the average or median value, thereby reducing the color difference between the inside and the outside of the boundary of the region of interest 1.

In some embodiments, when there are at least two regions of interest 1 includes, each region of interest 1 may have its own corresponding second ultrasonic image, and the second beam-forming procedure for each second ultrasonic image may be the same or different.

Referring to an embodiment shown in FIG. 8, provided is an ultrasonic imaging method including the steps of:

Step S100: controlling the ultrasonic probe 10 to transmit ultrasonic waves to the biological tissue under examination 200.

Step S200: obtaining echo signals of the biological tissue under examination 200, wherein the echo signals comprise at least one group of channel data, and each group of channel data corresponds to signals output by an array element;

If the minimum processing area for receiving and reflecting ultrasonic waves in the biological tissue under examination 200 is referred to as a location point within the tissue, after reaching each location point of the biological tissue under examination 200, the ultrasonic waves may generate different reflections due to the different acoustic impedance of the tissue at different location points; then the reflected ultrasonic waves may be picked up by the receiving array elements, and each receiving array element may receive ultrasonic echoes of a plurality of location points. The ultrasonic echoes of different location points received by each receiving array element may form different channel data; and multiple channel data output by each receiving array element may form a set of channel data corresponding to the receiving array element.

Step S300: beam-forming the channel data at the first group of beam-forming points by using the first beam-forming procedure to obtain the beam-formed data of the first group of beam-forming points, wherein the first group of beam-forming points correspond to respective location points within the biological tissue under examination 200 in the space covered by the ultrasonic waves.

In ultrasonic imaging, a frame of two-dimensional image is obtained by sequentially arranging several beam-forming points in a two-dimensional plane according to a spatial position relationship, and then performing such operations as envelope detection, dynamic range compression and digital scan conversion (DSC). The beam-forming points is a result of summing each channel data after phase compensation; and the beam-forming points herein correspond to the above-mentioned location points. The key of phase compensation is to determine the time sequence of ultrasonic echoes arriving at each array element, and the time sequence is determined by the spatial position (the spatial distance divided by the speed of sound is equal to the time).

In this embodiment, a variety of beam-forming procedures may be pre-stored including but not be limited to, a delayed and apodized summation algorithm, a minimum variance (MV) beam forming procedure, a coherent factor beam forming procedure, a incoherent beam forming procedure, or a frequency domain beam forming procedure, etc. The first beam-forming procedure is one selected from the plurality of pre-stored beam-forming procedures.

In some embodiments, the imaging setting of the current ultrasonic imaging can be detected after the user configures the imaging for ultrasonic imaging, and the first beam-forming procedure matching the imaging setting can be determined from the plurality of predetermined beam-forming procedures, that is, an appropriate beam-forming procedure can be automatically recommended. The above-mentioned imaging setting may include but not be limited to the probe type of the ultrasonic probe 10, the scan imaging mode of the ultrasonic probe 10, the scene mode and the imaging parameter of ultrasonic imaging; wherein the probe type may include but not be limited to high-frequency probe, low-frequency probe, one-dimensional probe, two-dimensional probe, etc. The probe scan mode may include but not be limited to a linear scan mode, a convex scan mode, a sector scan mode, a deflection scan mode, etc. The type of biological tissue under examination may include but not be limited to a small organ, nerve, etc. The imaging parameters may include but not be limited to frequency, aperture, focus, transmission line, receiving line, etc.

In addition to being completely automatically selected by the imaging apparatus, in some embodiments, at least one beam-forming selection item may be displayed on the display interface after detecting that the imaging setting of the ultrasonic imaging is completed, each beam-forming selection item is linked to a beam-forming procedure; and when detecting the first selection instruction generated based on the user's selection instruction on the beam-forming selection items, the selected first beam-forming procedure may be invoked based on the first selection instruction. For example, as shown in FIG. 2, the user may select a current scene from the display interface as examining an adult heart, and the probe type is P4-2, the relevant beam-forming items may be displayed for the user to select.

Step S400: generating a first ultrasound image of the biological tissue under examination 200 from the beam-formed data of the first group of beam-forming points.

That is, the first ultrasonic image is an ultrasonic image about a region generated by using the first beam-forming procedure, where the region is defined as an entire imaging region (FFOV) hereinafter.

Step S500: determining the region of interest 1 in the first ultrasonic image.

The shape of the region of interest 1 may be regular or irregular, e.g. regular in FIG. 3 and irregular in FIG. 4, and the way in which the region of interest 1 is determined may be automatic or non-automatic. The automatic way may include but not be limited to: determining the region of interest 1 in the first ultrasonic image by image recognition and other techniques; for example, performing feature extraction on the first ultrasonic image to obtain features of the entire image, and then performing matching detection on the features of the image to obtain one or more matched region as the region(s) of interest 1. The non-automatic way may include but not be limited to: selecting the region of interest 1 on the first ultrasonic image by a manual operation by the user; for example, selecting one or more regions of interest 1 by means of gestures, peripherals, voice controls, or the like from the first ultrasonic image which has been outputted on the display interface.

Step S600: beam-forming the channel data at a second group of beam-forming points by using the second beam-forming procedure to obtain the beam-formed data of the second group of beam-forming points corresponding to respective position points in the region of interest 1 in a one-to-one correspondence.

The first beam-forming procedure is different from the second beam-forming procedure. The difference therebetween may include that the first beam-forming procedure and the second beam-forming procedure are two completely different algorithms, for example, the first beam-forming procedure uses coherence factor while the second beam-forming procedure uses delayed multiplication and summation. The difference may further include that the first and second beam-forming procedures use the same technique but different synthesis parameters, for example, the first beam-forming procedure and the second beam-forming procedure both use a conventional DAS but adopt different window functions. For the conventional DAS method, an apodized window curve is usually preset, including a rectangular window, a Gaussian window, a Hanning window, a semi-circular window, etc. Different window functions can obtain different image effects, for example, the image corresponding to the rectangular window has a high spatial resolution but a lot of clutter, while the image corresponding to the Hanning window suppresses clutter but has a low spatial resolution; accordingly, the use of different window functions can also be considered as two different beam-forming procedures.

The second beam-forming procedure mentioned above is suitable for the imaging of region of interest 1, which can well represent the details of region of interest 1. In some embodiments, the selection of the second beam-forming procedure may be similar to the first beam-forming procedure, i.e. at least one beam-forming selection item is displayed for the user to select on the display interface, each beam-forming selection item is linked to a beam-forming procedure, a second selection instruction generated based on the user's selection instruction on the beam-forming selection items may be detected to invoke a second beam-forming procedure based on the second selection instruction. Different from the first beam-forming procedure, the beam-forming selection items displayed on the display interface can be determined based on the current imaging setting; alternatively, tissue structure within the region of interest 1 in the first ultrasonic image can be recognized after the determination of the region of interest 1, and the second beam-forming procedure can be determined from the plurality of predetermined beam-forming procedures based on the tissue structure within the region of interest 1. Furthermore, independent of the operation of the user, the it is also possible to determine which second beam-forming procedure to use directly from the recognized tissue structure of the region of interest 1, For example, the tissue structure contained in the region of interest 1 on the left in FIG. 5 is the tissue boundary, some beam-forming procedures being able to enhance the boundary information in the resultant ultrasound image may be used as the second beam-forming procedure. For another example, the tissue structure within the region of interest 1 on the right in FIG. 5 is small tissues, then the beam-forming procedures being able to improve the spatial resolution of the resultant ultrasound image can be used as the second beam-forming procedure.

Step S700: generating the second ultrasonic image of the region of interest 1 according to the beam-formed data of the second group of beam-forming points.

Step S800: fusing and displaying the first ultrasonic image and the second ultrasonic image.

The ultrasonic image of the whole imaging region and the ultrasonic image of region of interest 1 are fused, thereby taking into account the overall and local imaging effects. When the display 70 displays the fused image, the user can not only grasp the entire imaging region from the original first ultrasonic image, but also better understand the characteristics of the tissue structure within the region of interest 1 based on the original second ultrasonic image.

In some embodiments, the fusion between the first ultrasonic image and the second ultrasonic image is a “segmented” fusion which may be implemented in the following specific ways. One way may be to directly overlay the second ultrasonic image on the region of interest 1 in the first ultrasonic image, so that the region of interest 1 may directly display the second ultrasonic image. Another way may be to segment the part other than the region of interest 1 from the first ultrasonic image, and splice the second ultrasonic image with the part other than the region of interest 1 in the first ultrasonic image to obtain the fused image.

In other embodiments, the fusion between the first ultrasonic image and the second ultrasonic image is a “non-segmented” fusion. For example, the following formula may be used to fuse the first may image and the second may image to obtain the fused image:

P(i)ₒᵤₜₚᵤₜ=α(i)_{local}×P(i)_{local}+β(i)_{FFOV}×P(i)_{FFOV},i=1,2 . . . N

where P(i)_{local} represents a pixel value corresponding to the ith location point in the second ultrasonic image, α(i)_{local} is a second fusion coefficient corresponding to the ith location point, P(i)_{FFOV} represents a pixel value corresponding to the ith location point in the first ultrasonic image, β(i)_{FFOV} is a first fusion coefficient corresponding to the ith location point, and P(i)ₒᵤₜₚᵤₜ represents a pixel value corresponding to the ith location point in the fusion image. Generally, the sum of the fusion coefficient α(i)_{local} and β(i)_{FFOV} is 1, but it can also be other values. The fusion coefficient may be either a real number or a complex number; and one of the two fusion coefficients may be 0 or 1, or both may be 0 or 1. When the second fusion coefficient is 1 and the first fusion coefficient is 0, the imaging effect is consistent with the above-mentioned “segmented” fusion image.

In some embodiments, as shown in FIG. 7, in order to make the boundary of the region of interest 1 more natural, a transition zone 2 may also be generated around the region of interest 1, and the pixel values within the transition zone 2 may be filled according to the pixel values in the region of interest 1. For example, an average or median value of the pixel values within the entire region of interest 1 may be obtained and then the transition zone 2 may be filled with the average or median value, thereby reducing the color difference between the inside and the outside of the boundary of the region of interest 1.

In some embodiments, when there are at least two regions of interest 1 includes, each region of interest 1 may have its own corresponding second ultrasonic image, and the second beam-forming procedure for each second ultrasonic image may be the same or different.

In this example, the first ultrasonic image and the second ultrasonic image used for fusion are obtained based on the echo signals of the same ultrasonic waves, and the fusion process thereof is shown in FIG. 6. That is to say, after the ultrasonic probe 10 generates ultrasonic waves to the biological tissue under examination 200, channel data is obtained according to the echo signals. The channel data can be stored in addition to the beam-formed data of the first group of beam-forming points. After determining the region of interest 1, the beam-formed data of the second group of beam-forming points is obtained according to the same channel data.

In some embodiments, the process of obtaining the beam-formed data of the second group of beam-forming points may be as follows: beam-forming the channel data at a third group of beam-forming points by using the second beam-forming procedure to obtain beam-formed data of the third group of beam-forming points, the third group of beam-forming points corresponding to respective location points in the biological tissue under examination 200 in a space covered by the ultrasonic waves; and selecting data corresponding to location points falling within the region of interest 1 from the third group of beam-forming points as the beam-formed data of the second group of beam-forming points. That is to say, the channel data is synthesized by the second beam-forming procedure first, and then the data of the beam-forming points corresponding to the location points in the region of interest 1 is selected as the beam-formed data of the second group of beam-forming points.

In other embodiments, after the region of interest 1 is determined, it is also possible to transmit new ultrasonic waves to the region of interest 1. To avoid confusion, the ultrasonic wave transmitted to the entire imaging region (the biological tissue under examination 200) is referred to as the first ultrasonic wave, the echo of the first ultrasonic wave is referred to as the first echo signal, the channel data extracted from the first echo signal is referred to as the first channel data, then the beam-formed data of the first group of beam-forming points is obtained by beam-forming of the first channel data; whilst the ultrasonic wave re-emitted to the region of interest 1 is referred to as the second ultrasonic wave, the ultrasonic probe 10 may receive the second echo signal returned by the biological tissue under examination 200 in the region of interest 1 after the second ultrasonic wave is transmitted to the region of interest 1, the second channel data can be extracted from the second echo signal and then be beam synthesized at the second group of beam-forming points by using the second beam-forming procedure to obtain the beam-formed data of the second group of beam-forming points; and finally, a second ultrasonic image is generated according to the beam-forming data of the second group of beam-forming points. It can thus be seen that, in other embodiments, the user may re-transmit the second ultrasonic wave for the region of interest 1, and the angle and direction of the second ultrasonic wave can be different from those of the first ultrasonic wave, so the second ultrasonic wave that is more appropriate for the region of interest 1 may be selected to further increase the imaging effect.

Furthermore, how to transmit the second ultrasonic wave can be determined according to the region of interest 1. In some embodiments, after determining the region of interest 1, the processor 50 may calculate data about the length and width of the region of interest 1, determine the transmitting array elements and the transmitting beam parameters according to the data about the length and width of the region of interest 1, and then control the transmitting array elements on the ultrasonic probe 10 to transmit the second ultrasonic wave to the region of interest 1 according to the transmitting beam parameters.

In the above-mentioned embodiments, the first beam-forming procedure is used to generate the first ultrasonic image for the entire imaging region, and the second beam-forming procedure is used to generate the second ultrasonic image for the region of interest, and finally the two images are fused and displayed. In addition, the first beam-forming procedure can be selected automatically by the apparatus or the user; and the second beam-forming can be selected according to the tissue information in the region of interest, so that more suitable beam-forming procedures can be used correspondingly.

The present disclosure is illustrated with reference to various exemplary embodiments. However, those skilled in the art may recognize that the exemplary embodiments can be changed and modified without departing from the scope of the present disclosure. For example, various operation steps and components used to execute the operation steps may be implemented in different ways (for example, one or more steps may be deleted, modified, or combined into other steps) according to specific application(s) or any number of cost functions associated with the operation of the system.

In addition, as understood by those skilled in the art, the principles herein may be reflected in a computer program product on a computer-readable storage medium that is preloaded with computer-readable program code. Any tangible, non-temporary computer-readable storage medium can be used, including magnetic storage devices (hard disks, floppy disks, etc.), optical storage devices (CD-ROMs, DVDs, Blu Ray disks, etc.), flash memory and/or the like. The computer program instructions may be loaded onto a general purpose computer, a special purpose computer, or other programmable data processing device to form a machine, so that these instructions executed on a computer or other programmable data processing device can form a device that realizes a specified function. These computer program instructions may also be stored in a computer-readable memory that can instruct a computer or other programmable data processing device to run in a specific way, so that the instructions stored in the computer-readable memory can form a manufacturing product, including a realization device to achieve a specified function. The computer program instructions may also be loaded onto a computer or other programmable data processing device to execute a series of operating steps on the computer or other programmable device to produce a computer-implemented process, so that instructions executed on the computer or other programmable device can provide steps for implementing a specified function.

Although the principles herein have been shown in various embodiments, many modifications to structures, arrangements, proportions, elements, materials, and components that are specifically adapted to specific environmental and operational requirements may be used without deviating from the principles and scope of the present disclosure. These and other modifications and amendments will be included in the scope of the present disclosure.

The foregoing specific description has been illustrated with reference to various embodiments. However, those skilled in the art will recognize that various modifications and changes can be made without departing from the scope of the present disclosure. Accordingly, the present disclosure is illustrative rather than restrictive, and all such modifications will be included in its scope. Similarly, there are solutions to these and other advantages and problems of the various embodiments as described above. However, the benefits, the advantages, solutions to problems, and any elements that can produce them or make them more explicit should not be interpreted as critical, required, or necessary one. The term “comprise” and any other variations thereof used herein are non-exclusive; accordingly, a process, method, article or device that includes a list of elements may include not only these elements, but also other elements that are not explicitly listed or are not part of said process, method, article or device. In addition, the term “coupling” and any other variations thereof as used herein may refer to physical, electrical, magnetic, optical, communication, functional, and/or any other connection.

Those skilled in the art will realize that many changes can be made to the details of the above embodiments without departing from the basic principles of the present disclosure. The scope of the present disclosure shall therefore be determined in accordance with the following claims.

What is claimed is:

1. An ultrasonic imaging method, comprising:
controlling an ultrasonic probe to transmit ultrasonic waves to a biological tissue under examination and receive echoes from the biological tissue under examination to obtain multiple groups of channel data;
beam-forming the channel data at a first group of beam-forming points by using a first beam-forming procedure to obtain beam-formed data of the first group of beam-forming points, the first group of beam-forming points corresponding to respective location points in the biological tissue under examination in a space covered by the ultrasonic waves;
generating a first ultrasonic image of the biological tissue under examination according to the beam-formed data of the first group of beam-forming points;
determining a region of interest in the first ultrasonic image;
beam-forming the channel data at a second group of beam-forming points by using a second beam-forming procedure to obtain beam-formed data of the second group of beam-forming points, the second group of beam-forming points corresponding to respective location points in the region of interest;
generating a second ultrasonic image of the region of interest according to the beam-formed data of the second group of beam-forming points; and
displaying the first ultrasonic image and the second ultrasonic image in a fusion manner.

2. The ultrasonic imaging method according to claim 1, wherein said beam-forming the channel data at a second group of beam-forming points using a second beam-forming procedure to obtain beam-formed data of the second group of beam-forming points comprises:
beam-forming the channel data at a third group of beam-forming points by using the second beam-forming procedure to obtain beam-formed data of the third group of beam-forming points, the third group of beam-forming points corresponding to respective location points in the biological tissue under examination in a space covered by the ultrasonic waves; and
selecting data corresponding to location points falling within the region of interest from the third group of beam-forming points as the beam-formed data of the second group of beam-forming points.

3. The method according to claim 1, before beam-forming the channel data at the first group of beam-forming points using the first beam-forming procedure, further comprising:
obtaining an imaging setting of current ultrasonic imaging, and determining a beam-forming procedure matching the imaging setting from a plurality of predetermined beam-forming procedures as the first beam-forming procedure based on the imaging setting; or
displaying a plurality of beam-forming selection items on a display interface, each beam-forming selection item being associated with at least one beam-forming procedure; and detecting a first selection instruction generated based on a user's selection instruction on the beam-forming selection items to determine the first beam-forming procedure based on the first selection instruction.

4. The method according to claim 3, wherein the imaging setting comprises at least one of an ultrasonic probe type, a probe scan mode, a type of biological tissue under examination, and an imaging parameter for ultrasonic imaging.

5. The method according to claim 1, before said beam-forming the channel data at a second group of beam-forming points using a second beam-forming procedure, further comprising:
determining the second beam-forming procedure from a plurality of predetermined beam-forming procedures based on a region image within the region of interest in the first ultrasonic image; or
displaying a plurality of beam-forming selection items on a display interface after determining the region of interest, each beam-forming selection item being associated with at least one beam-forming procedure; and detecting a second selection instruction generated based on a user's selection instruction on the beam-forming selection items to determine the second beam-forming procedure based on the second selection instruction.

6. The method according to claim 5, wherein said determining the second beam-forming procedure from a plurality of predetermined beam-forming procedures based on a region image within the region of interest in the first ultrasonic image comprises:
obtaining tissue information contained in the region image within the region of interest in the first ultrasonic image, and determining the second beam-forming procedure from the plurality of predetermined beam-forming procedures based on the tissue information.

7. The method according to claim 6, wherein said determining the second beam-forming procedure from the plurality of predetermined beam-forming procedures based on the tissue information comprises:
determining a beam-forming procedure being able to enhance boundary information as the second beam-forming procedure when the region image within the region of interest in the first ultrasonic image contains more tissue boundaries than small tissues; or
determining a beam-forming procedure being able to improve spatial resolution as the second beam-forming procedure when the region image within the region of interest in the first ultrasonic image contains more small tissues than tissue boundaries.

8. The method according to claim 3, wherein the plurality of predetermined beam-forming procedures comprise at least two of a delay and sum beam forming procedure, a minimum variance beam forming procedure, a coherent factor beam forming procedure, an incoherent beam forming procedure, and a frequency domain beam forming procedure.

9. The method according to claim 1, wherein said displaying the first ultrasonic image and the second ultrasonic image in a fusion manner comprises:
overlaying the second ultrasonic image on the region of interest in the first ultrasonic image; or
segmenting a part outside the region of interest from the first ultrasonic image, and displaying the second ultrasonic image with the part outside the region of interest from the first ultrasonic image in a spliced manner.

10. The method according to claim 1, wherein the first beam-forming procedure differs from the second beam-forming procedure in at least one of principles, steps and parameters.

11. An ultrasonic imaging method, comprising:
controlling an ultrasonic probe to transmit first ultrasonic waves to a biological tissue under examination and receive echoes from the biological tissue under examination to obtain a first channel data;
beam-forming the first channel data at a first group of beam-forming points using a first beam-forming procedure to obtain beam-formed data of the first group of beam-forming points, the first group of beam-forming points corresponding to respective location points in the biological tissue under examination in a space covered by the ultrasonic waves;
generating a first ultrasonic image of the biological tissue under examination according to the beam-formed data of the first group of beam-forming points;
determining a region of interest based on the first ultrasonic image;
controlling the ultrasonic probe to transmit second ultrasonic waves to the region of interest and receive echoes from the region of interest to obtain a second channel data;
beam-forming the second channel data at a second group of beam-forming points using a second beam-forming procedure to obtain beam-formed data of the second group of beam-forming points, the second group of beam-forming points corresponding to respective location points in the region of interest;
generating a second ultrasonic image according to the beam-formed data of the second group of beam-forming points; and
displaying the first ultrasonic image and the second ultrasonic image in a fusion manner.

12. The method according to claim 11, before beam-forming the channel data at the first group of beam-forming points using the first beam-forming procedure, further comprising:
obtaining an imaging setting of current ultrasonic imaging, and determining a beam-forming procedure matching the imaging setting from a plurality of predetermined beam-forming procedures as the first beam-forming procedure based on the imaging setting; or
displaying a plurality of beam-forming selection items on a display interface, each beam-forming selection item being associated with at least one beam-forming procedure; and detecting a first selection instruction generated based on a user's selection instruction on the beam-forming selection items to determine the first beam-forming procedure based on the first selection instruction.

13. The method according to claim 12, wherein the imaging setting comprises at least one of an ultrasonic probe type, a probe scan mode, a type of biological tissue under examination, and an imaging parameter for ultrasonic imaging.

14. The method according to claim 11, before said beam-forming the channel data at a second group of beam-forming points using a second beam-forming procedure, further comprising:
determining the second beam-forming procedure from a plurality of predetermined beam-forming procedures based on a region image within the region of interest in the first ultrasonic image; or
displaying a plurality of beam-forming selection items on a display interface after determining the region of interest, each beam-forming selection item being associated with at least one beam-forming procedure; and detecting a second selection instruction generated based on a user's selection instruction on the beam-forming selection items to determine the second beam-forming procedure based on the second selection instruction.

15. The method according to claim 14, wherein said determining the second beam-forming procedure from a plurality of predetermined beam-forming procedures based on a region image within the region of interest in the first ultrasonic image comprises:
obtaining tissue information contained in the region image within the region of interest in the first ultrasonic image, and determining the second beam-forming procedure from the plurality of predetermined beam-forming procedures based on the tissue information.

16. The method according to claim 15, wherein said determining the second beam-forming procedure from the plurality of predetermined beam-forming procedures based on the tissue information comprises:
determining a beam-forming procedure being able to enhance boundary information as the second beam-forming procedure when the region image within the region of interest in the first ultrasonic image contains more tissue boundaries than small tissues; or
determining a beam-forming procedure being able to improve spatial resolution as the second beam-forming procedure when the region image within the region of interest in the first ultrasonic image contains more small tissues than tissue boundaries.

17. The method according to claim 11, wherein said displaying the first ultrasonic image and the second ultrasonic image in a fusion manner comprises:
overlaying the second ultrasonic image on the region of interest in the first ultrasonic image; or
segmenting a part outside the region of interest from the first ultrasonic image, and displaying the second ultrasonic image with the part outside the region of interest from the first ultrasonic image in a spliced manner.

18. The method according to claim 11, wherein the first beam-forming procedure differs from the second beam-forming procedure in at least one of principles, steps and parameters.

19. An ultrasonic imaging apparatus, comprising:
an ultrasonic probe configured to transmit ultrasonic waves to a biological tissue under examination and receive echoes of the biological tissue under examination to obtain channel data;
a beam former configured to beam-forming the channel data to obtain beam-formed data;
a display configured to display an ultrasonic image; and
a processor configured to control the ultrasonic probe, the beam former and the display to:
transmit ultrasonic waves to a biological tissue under examination and receive echoes from the biological tissue under examination to obtain multiple groups of channel data;
perform a beam-forming on the channel data at a first group of beam-forming points by using a first beam-forming procedure to obtain beam-formed data of the first group of beam-forming points, the first group of beam-forming points corresponding to respective location points in the biological tissue under examination in a space covered by the ultrasonic waves;
generate a first ultrasonic image of the biological tissue under examination according to the beam-formed data of the first group of beam-forming points;
determine a region of interest in the first ultrasonic image;
perform a beam forming on the channel data at a second group of beam-forming points by using a second beam-forming procedure to obtain beam-formed data of the second group of beam-forming points, the second group of beam-forming points corresponding to respective location points in the region of interest; or, transmit second ultrasonic waves to the region of interest and receive echoes from the region of interest to obtain a second channel data, and perform a beam-forming on the second channel data at a second group of beam-forming points using a second beam-forming procedure to obtain beam-formed data of the second group of beam-forming points, the second group of beam-forming points corresponding to respective location points in the region of interest;
generating a second ultrasonic image of the region of interest according to the beam-formed data of the second group of beam-forming points; and
displaying the first ultrasonic image and the second ultrasonic image in a fusion manner.
